(19) United States
(12) Patent Application Publication
(10) Pub. No.: US 2025/0366818 A1
(43) Pub. Date: Dec. 4, 2025

(54) Method and Apparatus of Intelligent Analysis for Liver Tumor

(71) Applicants: Hsiao-Ching Nien, Taipei City (TW); YUAN High-Tech Development Co., Ltd., Taipei City (TW)

(72) Inventors: Hsiao-Ching Nien, Taipei City (TW); Ta-Hsiang Lin, New Taipei City (TW); Pei-Lien Chou, Taipei City (TW)

(21) Appl. No.: 19/298,422

(22) Filed: Aug. 13, 2025

Related U.S. Application Data
(62) Division of application No. 18/615,351, filed on Mar. 25, 2024.

(30) Foreign Application Priority Data
Nov. 21, 2019 (TW) 108142298

(51) Int. Cl. A61B 8/08; A61B 8/00; G06T 7/00; G06V 10/764; G06V 10/774; G06V 20/70; G16H 15/00; G16H 30/40; G16H 50/30

(52) U.S. Cl. CPC A61B 8/085; A61B 8/4427; A61B 8/463; A61B 8/54; G06T 7/0014; G06V 10/764; G06V 10/774; G06V 20/70; G16H 15/00; G16H 30/40; G16H 50/30; G06T 2207/10132; G06T 2207/20081; G06T 2207/30056; G06T 2207/30096; G06V 2201/031

(57) ABSTRACT

Provided is a method and apparatus of intelligent analysis for liver tumors, including an analysis module for receiving YOLOR-based training to acquire sufficient intelligence to detect and locate liver tumor automatically and attain a mAP score as high as 0.56 required to distinguish lesions of benignant and malignant liver tumors in medical images from each other, attaining a mAP score of 0.628 for tumors at least 5 cm in size or a mAP score of 0.33 for tumors less than 5 cm in size. Thus, the area under the liver tumor differentiation curve of the analysis module and the mAP score reach 0.9 and 0.56 respectively. The values equal those of the effect of the diagnosis rate of liver tumors with CT and MRI in practice. The method is advantageous in terms of higher speed and thus can diagnose liver tumors earlier, preclude delays and radiation, but incur low cost.

### TECHNICAL FIELD OF THE INVENTION

The present invention relates to a method and apparatus for analyzing a liver tumor, and particularly to a novel, real-time artificial intelligence for coordinating ultrasonography with a deep learning algorithm to automatically detect and locate a liver tumor and determine in real time whether the liver tumor is malignant or benign according to a large dataset, with an mAP score as high as 0.56 in identifying the liver tumor, allowing a categorizer model of the present invention to attain a high-precision standard similar to that of a CT or MRI.

### DESCRIPTION OF THE RELATED ARTS

Liver cancer is the fourth worldwide death cause. The most common causes of liver cancer in Asia are hepatitis B virus and hepatitis C viruses and aflatoxin. The hepatitis C virus is a common cause in the United States and Europe. The liver cancers caused by steatohepatitis, diabetes, and triglyceride have become increasingly serious.

Surgery is currently the most direct method for treating liver cancers. However, early liver cancer diagnoses and postoperative patient-related prognostic indicators are also very important. A patient having a liver cancer confirmed by early diagnosis usually have more treatment options, where the treatment efficacy is shown by an improved survival rate of patients. Therefore, regular inspection and early diagnosis and treatment are the keys to improve the quality of life and to prolong the survival rate of patients.

In addition to early diagnoses including liver function blood test, hepatitis B virus and hepatitis C virus infection, and alpha-fetoprotein, abdominal ultrasound is an important test for liver disease, as studies indicated. An early study denoted that the liver blood tests of ⅓ patients with small HCC remained normal indexes for alpha-fetoprotein. Ultrasound examination must be complemented for early detection of liver cancer. Furthermore, abdominal ultrasound examination has the features of quickness, easiness and non-radiation, which becomes an important tool for screening liver cancer.

The diagnosis of liver cancer is different from those of other cancers. Its confirmation does not require biopsy, but is directly obtained through imaging diagnosis like abdominal ultrasound, computed tomography (CT), and magnetic resonance imaging (MRI), etc. Its sensitivity and specificity are 0.78˜0.73 and 0.89˜0.93, 0.84˜0.83 and 0.99˜0.91, and 0.83 and 0.88, respectively.

Ultrasonography is convenient, but has its own limit. For example, operator experience, patient obesity, existence of liver fibrosis or cirrhosis, etc. would affect the accuracy of ultrasound. Therefore, when malignancy is detected out through ultrasonography, a second imaging detection would be arranged, like CT or assisted diagnosis of MRI. Yet, these two detections have expensive costs for health care and lengthy examination schedules; and CT has consideration on more radiation exposure.

There has never been any large-scale research about performing automatic detection and diagnosis of malignant liver tumors, mainly HCC, through deep learning (DL). Hence, the prior arts do not fulfil all users' requests on actual use.

### SUMMARY OF THE INVENTION

The present invention provides a novel, real-time artificial intelligence for coordinating ultrasonography with a deep learning algorithm to automatically detect and locate a liver tumor and determine in real time whether the liver tumor is malignant or benign according to a large dataset, with a mAP score as high as 0.56 in identifying the liver tumor, allowing a categorizer model of the present invention to attain a high-precision standard similar to that of a CT or MRI and thus provide physicians with radiation-free and safe ultrasonography to rapidly and accurately diagnose liver tumor categories.

To achieve the above purpose, the present invention is a method of intelligent analysis (IA) for liver tumor, comprising steps of: (a) first step: providing a device of ultrasonography to scan an area of liver of an examinee from an external position to obtain an ultrasonic image of a target liver tumor of the examinee; (b) second step: obtaining a plurality of existing ultrasonic reference images of benignant and malignant liver tumors; (c) third step: obtaining a plurality of liver tumor categories from the existing ultrasonic reference images based on the shading and shadowing areas of the existing ultrasonic reference images to mark a plurality of tumor pixel areas in the existing ultrasonic reference images and identify the liver tumor categories of the tumor pixel areas, and its test flow involves using an AI module of a You Only Learn One Representation (YOLOR) to perform automatic lesion detection with classification on liver tumor images of abdominal ultrasound, wherein the YOLOR-based AI module detects and locates liver tumors automatically and in real time, determines whether the liver tumors are benignant or malignant, and then generates an AI result; (d) fourth step: obtaining the tumor pixel areas in the ultrasonic reference images to train a categorizer model with the coordination of a deep learning algorithm, and its train flow entails introducing thousands of existing ultrasonic reference images showing benignant and malignant liver tumors and collected in Second step into the test flow of Third step, using the YOLOR-based AI module to compute the locations of the liver tumors and determine the nature of the benignant or malignant liver tumors to obtain an AI result, and then comparing the AI result with a clinician's markers to calculate loss and update weights; after that, the next ultrasonic reference image of liver tumors undergoes training, and thousands of instances of training are carried out in the aforesaid manner to allow the categorizer model to correct its intelligence level, wherein a mAP score is calculated at the end of the thousands of instances of the train flow, wherein, after the training, the highest mAP score thus calculated is 0.56, allowing this score to be for use in analyzing an ultrasonic image of a target liver tumor of the examinee; and (e) fifth step: processing an analysis of the ultrasonic image of the target liver tumor of the examinee with the categorizer model to provide the analysis to a clinician to determine the target liver tumor a liver tumor category and predict a risk probability of malignance of the target liver tumor.

### BRIEF DESCRIPTION OF THE DRAWINGS

The present invention will be better understood from the following detailed description of the preferred embodiment according to the present invention, taken in conjunction with the accompanying drawings, in which

FIG. 1 is the flow view showing the preferred embodiment according to the present invention; and

FIG. 2 is the block view.

FIG. 3 is a test flow view of performing automatic detection with classification on abdominal ultrasound images with an AI module of YOLOR according to the present invention.

FIG. 4 is a flow view of training a categorizer model according to the present invention.

### DESCRIPTION OF THE PREFERRED EMBODIMENT

The following description of the preferred embodiment is provided to understand the features and the structures of the present invention.

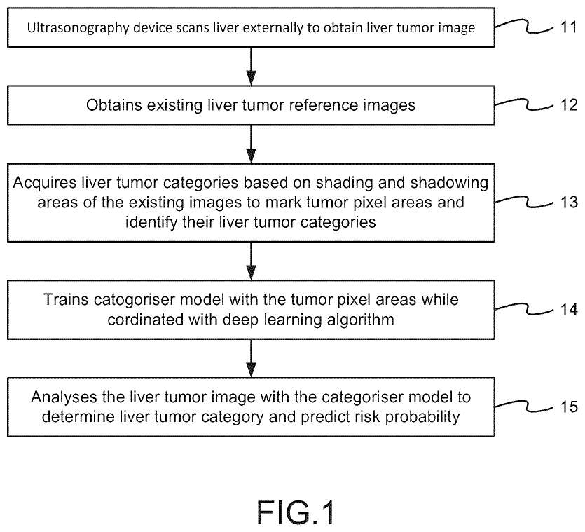

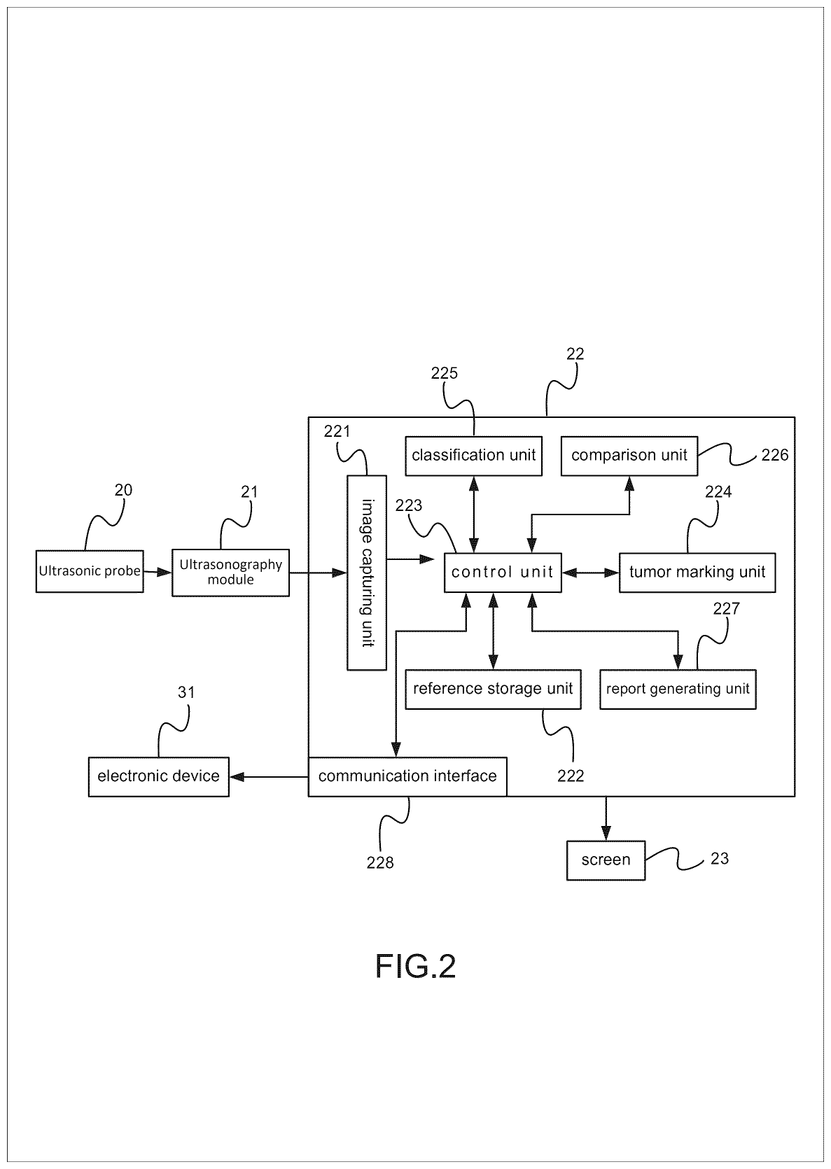

Please refer to FIG. 1 and FIG. 2, which are a flow view and a block view showing a preferred embodiment according to the present invention. As shown in the figures, the present invention is a method of intelligent analysis (IA) for liver tumor, comprising the following steps:

(a) First step 11: A device of ultrasonography is provided to scan an area of liver of an examinee from an external position to obtain an ultrasonic image of a target liver tumor of the examinee.

(b) Second step 12: A plurality of existing ultrasonic reference images of benignant and malignant liver tumors are obtained.

(c) Third step 13: Based on the shading and shadowing areas of the existing ultrasonic reference images, a plurality of liver tumor categories of the existing ultrasonic reference images are acquired to mark a plurality of tumor pixel areas in the existing ultrasonic reference images and identify the liver tumor categories of the tumor pixel areas, and its test flow involves using an AI module of a You Only Learn One Representation (YOLOR) to perform automatic lesion detection with classification on liver tumor images of abdominal ultrasound. The AI module of YOLOR detects and locates liver tumors automatically and in real time, determines whether the liver tumors are benignant or malignant, and then generates an AI result.

(d) Fourth step 14: The tumor pixel areas in the ultrasonic reference images is used to train a categorizer model with the coordination of a deep learning algorithm, and its train flow entails introducing thousands of existing ultrasonic reference images showing benignant and malignant liver tumors and collected in Second step 12 into the test flow of Third step 13, using the AI module of YOLOR to compute the locations of the liver tumors and determine the nature of the benignant or malignant liver tumors to obtain an AI result, and then comparing the AI result with a clinician's markers to calculate loss and update weights. After that, the next ultrasonic reference image of liver tumors undergoes training. Thousands of instances of training are carried out in the aforesaid manner to allow a categorizer model to correct its intelligence level. A mAP score is calculated at the end of the thousands of instances of the train flow. After the training, the highest mAP score thus calculated is 0.56. This score is for use in analyzing an ultrasonic image of a target liver tumor of the examinee. (e) Fifth step 15: An analysis of the ultrasonic image of the target liver tumor of the examinee is processed with the categorizer model to be provided to a clinician to determine the target liver tumor a liver tumor category and predict a risk probability of malignance of the target liver tumor. Thus, a novel method of IA for liver tumor is obtained.

The present invention uses an apparatus, comprising an ultrasonography module 21 and an analysis module 22.

The ultrasonography module 21 has an ultrasound probe 20.

The analysis module 22 connects to the ultrasonography module 21 and comprises an image capturing unit 221, a reference storage unit 222, a control unit 223, a tumor marking unit 224, a classification unit 225, a comparison unit 226, and a report generating unit 227. Therein, the control unit 223 is a central processing unit (CPU) processing calculations, controls, operations, encoding, decoding, and driving commands with/to the image capturing unit 221, the reference storage unit 222, the tumor marking unit 224, the classification unit 225, the comparison unit 226, and the report generating unit 227.

For applications, the present invention is practiced in a computer. The control unit 223 is a CPU of the computer; the tumor marking unit 224, the classification unit 225, the comparison unit 226, and the report generating unit 227 are programs and stored in a hard disk or a memory of the computer; the image capturing unit 221 is a digital visual interface (DVI) of the computer; the reference storage unit 222 is a hard drive; and the computer further comprises a screen 23, a mouse, and a keyboard for related input and output operations. Or, the present invention can be implemented in a server.

On using the present invention, an ultrasonic probe 20 of an ultrasonography module 21 provides emission of ultrasonography to an examinee from an external position corresponding to an area of liver to obtain an ultrasonic image of a target liver tumor of the examinee. During scanning, a physician may perceive at least one ultrasound image of a suspected tumor to be selected as an ultrasonic image of a target liver tumor.

By using an image capturing unit 221, an analysis module 22 obtains the ultrasound image of the target liver tumor of the examinee, where the image is formed through imaging with the ultrasonography module 21. A reference storage unit 222 stores a plurality of existing ultrasonic reference images of benignant and malignant liver tumors. A program is stored in an analysis module 22, where, on executing the program by a control unit 223, the program determines a liver tumor category to a clinician and predict a risk probability of malignance of the target liver tumor. The program comprises a tumor marking unit 224, a classification unit 225, a comparison unit 226, and a report generating unit 227.

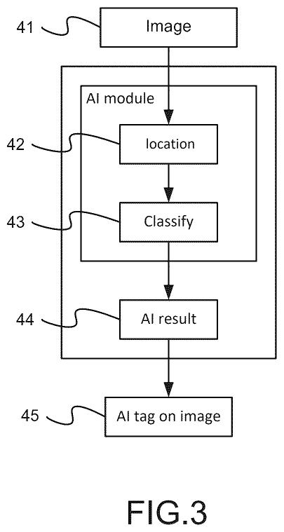

The tumor marking unit 224 obtains coefficients and/or parameters derived from empirical data to automatically mark pixel tumor areas in the ultrasonic reference images and identify a plurality of liver tumor categories. For example, the tumor marking unit 224 may process marking based on physician experiences. Specifically speaking, according to the present invention, the tumor marking unit 224 uses an AI module of You Only Learn One Representation (YOLOR) to perform automatic lesion detection with classification on liver tumor images of abdominal ultrasound, with its test flow shown in FIG. 3. In steps 41, 42, 43, liver tumor ultrasonic images are inputted to the AI module using YOLOR to detect and locate liver tumors automatically in real time, determine the nature of the benignant or malignant liver tumors, and then generate an AI result in step 44.

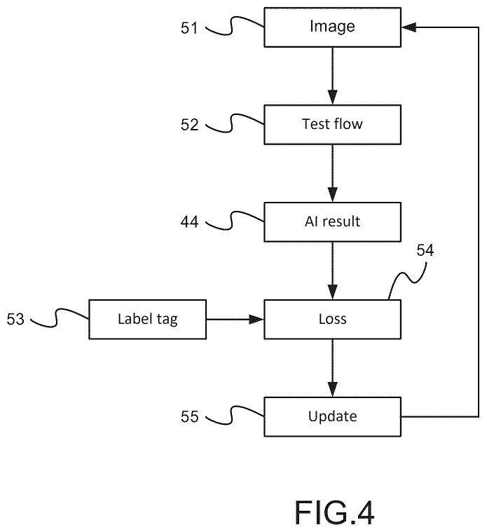

The classification unit 225 obtains the pixel tumor areas in the ultrasonic reference images to process training by using a deep learning algorithm to build a categorizer model. Specifically speaking, according to the present invention, the classification unit 225 performs train flow on a categorizer model, as shown in FIG. 4. In steps 51, 52, around 4000 reference liver tumor ultrasonic images are retrieved from existing ultrasonic reference images showing benignant and malignant liver tumors and collected in the reference storage unit 222 and introduced into the test flow shown in FIG. 3. Then, the AI module of YOLOR is used to compute the locations of the liver tumors and determine the nature of the benignant or malignant liver tumors to obtain an AI result in step 44. In step 45, the screen 23 displays AI markers on images. In step 53, the AI result and a clinician's markers are compared. Steps 54, 55 involve calculating loss and updating weights. After that, the next ultrasonic reference image of liver tumors undergoes training. Over 4,000 instances of training are carried out in the aforesaid manner to allow the categorizer model to correct its intelligence level and thus make increasingly precise determinations. After over 4,000 instances of the train flow have been carried out, a mAP score is calculated. After the training, the highest mAP score thus calculated is 0.56. This score is for use in analyzing an ultrasonic image of a target liver tumor of the examinee by the comparison unit 226.

The comparison unit 226 analyzes the ultrasonic image of the target liver tumor with the categorizer model to provide the clinician for determining the nature of the liver tumor of the examinee and further predicting a risk probability of malignance of the target liver tumor of the examinee. At last, the comparison unit 226 determines the liver tumor category and predicts the risk probability of malignance of the liver tumor by the clinician for the examinee to be inputted to the report generating unit 227 to produce a diagnostic report for assisting the physician in determining the nature of the liver tumor. The diagnostic report is directly displayed on the screen 23 or outputted via a communication interface 228 to an electronic device 31 for remote display thereon.

The present invention is the first of its kind to apply YOLOR to medical image recognition. As mentioned above, given YOLOR training, the analysis module gains sufficient intelligence to attain a mAP score as high as 0.56 required to distinguish lesions of benignant and malignant liver tumors in medical images from each other, attaining a mAP score of 0.628 for tumors at least 5 cm in size or a mAP score of 0.33 for tumors less than 5 cm in size. The abovementioned is the advantage achieved by the present invention, but the advantage is going to be augmented continuously through continuous training, and will even be augmented continuously because of increasingly smart AI modules in the future. Finally, images of diagnosing liver tumors according to golden criteria of CT, MRI or tissue biopsy are used. Thus, the area under the liver tumor differentiation curve of the analysis module and the mAP score reach 0.9 and 0.56 respectively. The values equal those of the effect of the diagnosis rate of liver tumors with CT and MRI in practice. However, the present invention is advantageous in terms of higher speed and thus can diagnose liver tumors earlier and preclude delays and radiation. Furthermore, the present invention incurs low equipment cost for the reasons explained below. According to the present invention, the analysis module that operates by AI master technology is connected to a PC-based ultrasound system equipped with probes so as to directly apply AI to image recognition, dispensing with complicated equipment, dispensing with the need to change the original PC-based ultrasound system, and dispensing with the need to alter any interfaces. All the present invention needs to do is send ultrasonic image data obtained by the PC-based ultrasound system to the analysis module so as to perform AI computation with a built-in AI module, dispensing with the need to access the resources of the original PC-based ultrasound system. Therefore, the present invention incurs low cost but performs computation fast. By contrast, AI computation performed according to prior art takes up performance otherwise exhibited by the original PC-based ultrasound system and thus reduces recognition speed, slowing down execution. The present invention is effective in performing AI judgement in real time, i.e., during a time period of only 10 frame delays±20%. The aforesaid results prove the high precision of a YOLOR-based analysis module in terms of detection and diagnosis. The aforesaid results can bring about the integration of automatic detection and diagnosis, provide a faster, more reliable screening reference to clinicians, and thereby enhance the efficiency and effectiveness of a diagnosis process, especially in the absence of abdominal ultrasonography specialists. The analysis module is unique in that it performs real-time examination with abdominal ultrasonography from the beginning to the end. The analysis module is the first of its kind to achieve the aforesaid results. More importantly, the imaging process of the analysis module using YOLOR is real-time, i.e., free of any delays. In addition, YOLOR is unique in terms of automatic detection and locating function with classification.

One of the difficulties in diagnosis of liver tumors is as follows: liver cancer is one of a small number of malignant diseases that do not necessarily require biopsy in order to be diagnosed but can be diagnosed solely through imaging diagnosis; and abdominal ultrasonic images lack definite locating criteria and borders, adding to the difficulty in AI learning and reading. The present invention enables experienced professionals working with a YOLOR-based analysis module to substitute for experienced abdominal ultrasonography clinicians. The present invention is effective in locating and classifying tumors automatically, performing reading correctly, and assisting experienced professionals with diagnosis.

Thus, the present invention uses the abundant experiences of abdominal ultrasound specialists as a base to mark a pixel area of a liver tumor in an ultrasound image. The parameters and coefficients of such empirical data are obtained for processing training by using the deep learning algorithm to obtain an mAP score as high as 0.56 for the liver tumor in the categorizer model. Hence, with the ultrasonography image, a help to the physician or ultrasound technician is immediately obtained through the present invention for determining the risk probability of malignance of the liver tumor and a base of reference is further provided for diagnosing the liver tumor category.

To sum up, the present invention is a method of IA for liver tumor, where ultrasonography is coordinated with a deep learning algorithm to determine the risk probability of malignant liver tumor; by using coefficients and/or parameters coordinated with empirical data, pixel tumor areas in ultrasonic reference images are marked out to obtain a categorizer model having an accuracy up to 86% through the deep learning algorithm; and, thus, physicians are assisted with radiation-free and safe ultrasonography to rapidly and accurately diagnose liver tumor categories.

The preferred embodiment herein disclosed is not intended to unnecessarily limit the scope of the invention. Therefore, simple modifications or variations belonging to the equivalent of the scope of the claims and the instructions disclosed herein for a patent are all within the scope of the present invention.

1. (canceled)

2. (canceled)

3. (canceled)

4. (canceled)

5. (canceled)

6. (canceled)

7. (canceled)

8. (canceled)

9. (canceled)

10. (canceled)

11. (canceled)

12. (canceled)

13. (canceled)

14. A method of analyzing a liver tumor comprising
ultrasonically scanning an area of a liver of an examinee from an external position and absent added contrast agent to obtain an ultrasonic image of a target liver tumor of said examinee;
obtaining, by an analysis module, a plurality of existing ultrasonic reference images of benign and of malignant liver tumors, wherein each existing reference image includes a clinician's marker of tumor pixel areas and liver tumor category for the reference image;
defining a result of a liver tumor category for one of the existing ultrasonic reference images based on shading and shadowing areas of the existing ultrasonic reference image and marking a plurality of tumor pixel areas in the existing ultrasonic reference image automatically with a you-only-learn-one-representation (YOLOR) module in the analysis module according to one or more parameters derived from empirical data locating and marking a plurality of tumor image pixel areas in said ultrasonic reference liver tumor image, wherein the liver tumor categories include benign liver tumors and malignant liver tumors;
training a categorizer model by comparing the automatically defined result of liver tumor category and marked tumor pixel areas in said ultrasonic reference image with the clinician's liver tumor category and marker of tumor pixel areas for the reference image with coordination of a learning algorithm and adjusting the one or more parameters of the analysis module based on the comparison;
iterating the defining and training for a next ultrasonic reference image and calculating a mean Average Precision (mAP) score until the mAP score meets or exceeds a mAP threshold; and
automatically analyzing said ultrasonic image of said target liver tumor of said examinee with said categorizer model and providing, in real time, a liver tumor category and a risk probability of malignance of said target liver tumor.

15. The method according to claim 14, wherein the liver tumor category and risk probability of malignance of said target liver tumor, as determined and predicted by said analysis module respectively, are directly displayed on a screen or outputted via a built-in communication interface to an electronic device for remote display thereon.

16. The method according to claim 14, wherein an area under a liver tumor differentiation curve of said analysis module reaches 0.9.

17. The method according to claim 14, wherein said analysis module attains a mAP score of 0.628 for tumors at least 5 cm in size.

18. The method according to claim 14, wherein said analysis module automatically analyzes the ultrasonic image of the target liver tumor of the examinee and provides the liver tumor category and risk probability of malignance of the target liver tumor within a time period of 10 frame delays±20%.

19. (canceled)
